(19) United States
(12) Patent Application Publication
(10) Pub. No.: US 2024/0156912 A1
(43) Pub. Date: May 16, 2024

(54) NOVEL METHOD

(71) Applicant: Babraham Institute, Cambridge (GB)

(72) Inventors: Adrian LISTON, Cambridge (GB); James DOOLEY, Cambridge (GB); Oliver BURTON, Cambridge (GB); Lubna KOUSER, Cambridge (GB); Ntombizodwa MAKUYANA, Cambridge (GB); Stephanie LIENART, Cambridge (GB)

(21) Appl. No.: 18/549,771

(22) PCT Filed: Mar. 10, 2022
(86) PCT No.: PCT/GB2022/050626

§ 371 Date: Sep. 8, 2023

(30) Foreign Application Priority Data
Mar. 10, 2021 (GB) 2103327.9

(51) Int. Cl. A61K 38/20; A61K 31/65; A61K 48/00; A61P 11/00; A61P 29/00

(52) U.S. Cl. CPC A61K 38/2013; A61K 31/65; A61K 48/0033; A61K 48/0058; A61K 48/0066; A61P 11/00; A61P 29/00

(57) ABSTRACT

The invention relates to a method of expanding a population of regulatory T cells in a tissue or organ of a subject, wherein said method comprises administration of IL-2 and a targeting moiety specific for said tissue or organ, and wherein said tissue or organ is the lung. The invention further relates to populations of regulatory T cells produced according to the method and the production of said population in vivo. Also provided is a pharmaceutical composition comprising IL-2 and a targeting moiety as defined herein as well as a method of treating a disease or disorder mediated by inflammation or for the reduction of inflammation which comprises the methods defined herein or administration of a pharmaceutical composition as defined herein.

### FIELD OF THE INVENTION

The invention relates to a method of expanding a population of regulatory T cells in a tissue or organ of a subject, wherein said method comprises administration of IL-2 and a targeting moiety specific for said tissue or organ, and wherein said tissue or organ is the lung. The invention further relates to populations of regulatory T cells produced according to the method and the production of said population in vivo. Also provided is a pharmaceutical composition comprising IL-2 and a targeting moiety as defined herein as well as a method of treating a disease or disorder mediated by inflammation or for the reduction of inflammation which comprises the methods defined herein or administration of a pharmaceutical composition as defined herein.

### BACKGROUND OF THE INVENTION

Acute Respiratory Distress Syndrome (ARDS) can be triggered through diverse infectious stimuli. Despite the multitude of potential triggers, in each case the pathologic process converges on several conserved inflammatory pathways leading to inappropriate inflammation. For example, severe type 1 inflammation in the lung can cause loss of lung function and is potentially fatal. It is involved in multiple non-infectious modalities, such as chronic obstructive pulmonary disease (COPD). However, severe respiratory infections are also immunopathologies which are driven by type 1 inflammation. Such infections include seasonal flu, H5N1 (avian flu), H1N1 (swine flu), SARS (severe acute respiratory syndrome) MERS (Middle East respiratory syndrome) and COVID-19. Responses to pandemics involving novel respiratory viruses typically include the development of new vaccines to prevent infection or the use of immunosuppression to prevent immunopathology in patients. Vaccines are highly effective and specific but due to their development time involve a considerable delay. In contrast, immunosuppression therapy uses generic/already known treatments, e.g. dexamethasone, so are quicker to put into use but due to their high level of generality optimal treatment is difficult to achieve because systemic immunosuppression blocks the development of a healthy immune response to the infecting virus.

There is therefore a great need for a therapy which provides a virus-agnostic approach which allows for rapid deployment and prevents pathology while still enabling antibody generation.

### SUMMARY OF THE INVENTION

According to a first aspect of the invention, there is provided a method of expanding a population of regulatory T cells in a tissue or organ of a subject in need thereof, wherein said method comprises administration of IL-2 and a targeting moiety specific for said tissue or organ, and wherein said tissue or organ is the lung.

According to a further aspect of the invention, there is provided a pharmaceutical composition comprising IL-2 and a targeting moiety specific for a tissue or organ of a subject, wherein said targeting moiety is specific for the lung.

According to a yet further aspect of the invention, there is provided a method of treating a disease or disorder mediated by inflammation and/or for the reduction of inflammation, wherein said method either comprises a method as defined herein or administering the pharmaceutical composition as defined herein to a subject in need thereof.

### BRIEF DESCRIPTION OF THE FIGURES

FIG. 1: Parabiotic analysis of lung regulatory T cell kinetics.

A) Parabiosis surgery was performed on Foxp3^{Thy1.1}CD45.1 and Foxp3^{Thy1.1}CD45.2 mice, with analysis of brain and blood CD4 T cells at 1, 2, 4, 8 and 12 weeks (n=12, 12, 18, 16, 14).

B) Curves of best fit for lung Tregs, showing CD69⁻ and CD69⁺ populations.

FIG. 2: Local expression of IL-2 drives a lung-specific expansion of regulatory T cells.

A) Wildtype littermates and Scgb1a1-ERT2^{Cre} RosaIL2 mice were treated with tamoxifen at day −14, to induce the production of IL-2 by bronchiolar non-ciliated club cells. Flow cytometry was used to assess the frequency of Foxp3⁺ cells within CD4 T cells in the blood, lung, spleen, lymph nodes and lung-draining mediastinal lymph nodes. n=5 mice/group.

B) 19-21 week old, male Scgb1a1-ERT2^{Cre} RosaIL2 mice were administered three doses of 0.2 mg/gram tamoxifen by oral gavage. After day 0 and day 12, organs were collected for flow analysis. Quantitative data for percentage of Tregs were gated in FlowJo and plotted using GraphPad prism. All data were analysed using Two Way Anova *P<0.05, **P<0.01, and ***P<0.001 was considered significant. n=5 animals/group.

C) 19-21 week old, male Scgb1a1-ERT2^{Cre} RosaIL2 mice were administered three doses of 0.2 mg/gram tamoxifen by oral gavage. After day 0, 1, 4, 7, 15, 21, 29 and 35, lungs were processed for qPCR Taqman analysis for IL-2. Values were extrapolated by making a standard curve first using the Thermofisher ProQuantum software and plotted using GraphPad prism. All data were analysed using Two Way Anova and T test at day 7; *P<0.05, **P<0.01, and ***P<0.001 was considered significant. n=5 animals/group.

D) Lymph nodes from the animals in C were also processed for qPCR Taqman analysis for IL-2. All data were analysed using Two Way Anova; *P<0.05, **P<0.01, and ***P<0.001 was considered significant. n=5 animals/group.

E) 19-21 week old, male Scgb1a1-ERT2^{Cre} RosaIL2 mice were administered three doses of 0.2 mg/gram tamoxifen by oral gavage. After day 0, 1, 4, 7, 15, 21, 29 and 35, lungs were processed for flow cytometric analysis. Data was analysed using FlowJo and percentage values obtained after gating for Foxp3+, CD4+, CD3⁺ cells and plotted using GraphPad prism. All data were analysed using Two Way Anova; *P<0.05, **P<0.01, and ***P<0.001 was considered significant. n=5 animals/group.

F) Lymph nodes from the animals in E were also processed for flow cytometric analysis and analysed in the same manner.

FIG. 3: Local expression of IL-2 protects against flu-induced neutrophil infiltration.

Wildtype littermates and Scgb1a1-ERT2^{Cre} RosaIL2 mice were treated with tamoxifen at day −14, to induce the production of IL-2 by bronchiolar non-ciliated club cells. Mice were then infected with intranasal mouse flu, and cohorts dissected at 0, 12 and 21 days post-infection. Flow cytometry was used to assess the frequency of neutrophils in the lungs of infected mice. n=5-6 mice/group.

FIG. 4: Intranasal administration of AAV6.2-mCC10-IL2 results in local expansion of regulatory T cells (Tregs) in the lungs.

12 week old, male mice were administered 10¹¹ (vg/ml) of the AAV6.2-mCC10-IL2 or PBS and at day 14 organs were collected for flow cytometric analysis. Data was analysed using FlowJo and percentage values obtained and plotted using GraphPad prism. All data were analysed using Two Way Anova between the groups; *P<0.05, **P<0.01, and ***P<0.001 was considered significant. n=5 animals/group.

### DETAILED DESCRIPTION OF THE INVENTION

According to a first aspect of the invention, there is provided a method of expanding a population of regulatory T cells in a tissue or organ of a subject in need thereof, wherein said method comprises administration of IL-2 and a targeting moiety specific for said tissue or organ, and wherein said tissue or organ is the lung.

In one embodiment, the methods defined herein comprise expanding a population of cells, such as a population of regulatory T cells. In a further embodiment, said expanding of a population of cells, such as a population of regulatory T cells, is in a tissue or organ of a subject in need thereof, such as a particular tissue or organ of interest.

References herein to the terms “expanding”, “expansion” and “expanded” or to the phrases “expanding a population of regulatory T cells” and “expanded population of regulatory T cells” include references to populations of cells which are larger than or comprise a larger number of cells than a non-expanded population. It will thus be appreciated that such an “expanded” population produced according to the methods defined herein comprises a larger number of cells than a population which has not been subjected to IL-2. Thus, in certain embodiments, the expanded population of cells produced according to the methods defined herein, such as an expanded population of regulatory T cells, comprises a larger number of cells compared to a reference population of cells. In one embodiment, the reference population of cells may be a population of cells not subjected to or administered with IL-2. In one embodiment, the expanded population of cells comprises a larger number of cells than the population prior to any administration of IL-2. In further embodiments, the reference population of cells may be located in a different tissue or organ to the expanded population of cells produced according to the methods defined herein. In a further embodiment, the expanded population of cells is an expanded population in a tissue or organ of a subject and comprises a larger number of cells compared to a population of cells not located in said tissue or organ of interest. For example, the expanded population of cells, in particular an expanded population of regulatory T cells, is expanded in the lung compared to a population of cells, in particular regulatory T cells, in the blood, spleen, liver and/or lymph nodes.

In one embodiment, the expanded population of cells produced according to the methods defined herein, such as an expanded population of regulatory T cells, comprises a population at least 2-fold, at least 3-fold, at least 4-fold, at least 5-fold, at least 6-fold, at least 7-fold, at least 8-fold, at least 9-fold, at least 10-fold, at least 11-fold, at least 12-fold, at least 13-fold, at least 14-fold or more larger than a population of cells which has not been subjected to or administered with IL-2. In a further embodiment, the expanded population of cells comprises a population at least 2-fold, at least 3-fold, at least 4-fold, at least 5-fold, at least 6-fold, at least 7-fold, at least 8-fold, at least 9-fold, at least 10-fold, at least 11-fold, at least 12-fold, at least 13-fold, at least 14-fold or more larger than a population of cells not located in the tissue or organ of interest. In a particular embodiment, the expanded population of cells is at least 2-fold, at least 4-fold, at least 5-fold, at least 6-fold, at least 7-fold, at least 8-fold, at least 12-fold, at least 13-fold or at least 14-fold larger than a reference population, such as a population of cells in the tissue or organ of interest which has not been subjected to or administered with IL-2 or a population of cells not located in the tissue or organ of interest. In some embodiments, the expanded population of cells comprises a larger proportion of cells which make up a subset of the population (e.g. a larger proportion of regulatory T cells within the total population of T cells in the tissue or organ). In further embodiments, the expanded population of cells is maximal at 21 days after administration, i.e. the population of cells is maximally expanded at 21 days after administration. In some embodiments, the expanded population of cells can be detected between 4 and 35 days after administration, i.e. expansion of the population of cells can be detected between 4 and 35 days after administration. Thus, in one embodiment the expanded population of cells/the expansion of the population of cells can be detected at 4 days after administration. In a further embodiment, the expanded population of cells/the expansion of the population of cells can be detected for 35 days after administration. In a yet further embodiment, maximal expansion of the population of cells can be detected at 21 days after administration.

Therefore, it will be appreciated that the expanded population of regulatory T cells as defined herein may be expanded in a manner which is dependent on the dose of IL-2 administered. Thus in certain embodiments, the expanded population of regulatory T cells as defined herein comprises a population which is larger than a reference population by a factor which is IL-2 dose-dependent.

In further embodiments, the expanded population of regulatory T cells produced according to the methods defined herein comprises a population of cells which have increased survival. Thus, in one embodiment, the expanded population of regulatory T cells produced according to the methods defined herein comprises increased survival. In a further embodiment, the expanded population of regulatory T cells comprises decreased, or reduced, cell death. In a yet further embodiment, the expanded population of regulatory T cells comprise increased proliferation. Thus, in one embodiment, the expanded population of regulatory T cells is larger than a reference population (e.g. a population of regulatory T cells not subjected to or administered with IL-2 or a population of cells not located in the tissue or organ of interest) because of increased survival of the expanded population of regulatory T cells. In a further embodiment, the expanded population of regulatory T cells is larger than a reference population because of decreased, or reduced, cell death in the expanded population of regulatory T cells. In a yet further embodiment, the expanded population of regulatory T cells is larger than a reference population because of increased proliferation. In a still further embodiment, the expanded population of regulatory T cells is larger than a reference population because of a combination of one or more of increased survival, decreased/reduced cell death and increased proliferation.

It will be appreciated that references herein to an “expanded population” produced according to the methods defined herein, such as an “expanded population of regulatory T cells”, may also include a population of cells which are activated. References herein to “expanding” may include the activation of a population of cells produced according to the methods defined herein, such as a population of regulatory T cells. Similarly, “expanding” also includes the expansion of an activated population of regulatory T cells, for example, a population which is already activated prior to administration of IL-2. Such activation of the population of cells produced according to the methods defined herein, such as a population of regulatory T cells, may be independent of an expansion or may be concomitant with an expansion of said population. Thus, in one embodiment, the expanded population of regulatory T cells comprises activated regulatory T cells. In a further embodiment, the expanded population of regulatory T cells is an activated population of regulatory T cells.

In an alternative embodiment, references herein to “expanding” or an “expanded population” produced according to the methods defined herein do not include activating said population or an activated population of cells. Thus, according to this embodiment, the expanded population of cells produced according to the methods defined herein, such as an expanded population of regulatory T cells, does not comprise an activated phenotype. In a further embodiment, the expanded population of regulatory T cells does not comprise activated regulatory T cells. Thus, in a yet further embodiment, the expanded population of regulatory T cells comprises the phenotype, such as the surface phenotype, of a population of regulatory T cells which have not been subjected to or administered with IL-2.

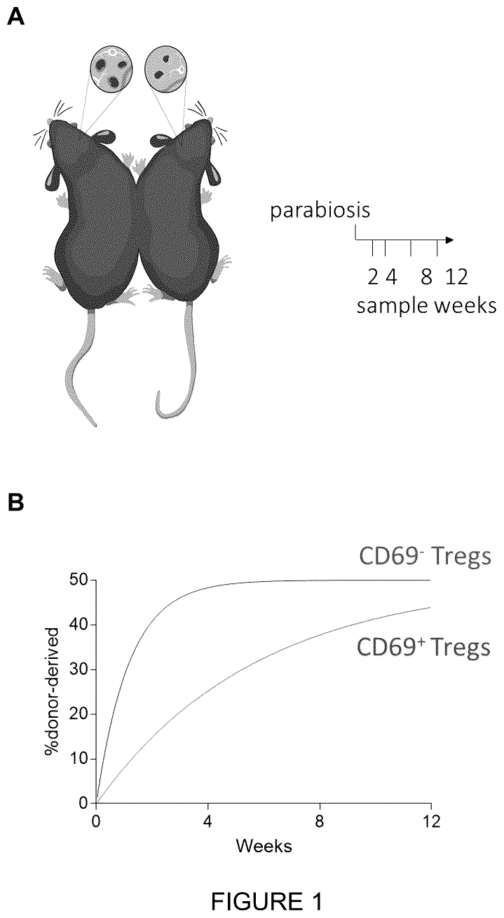

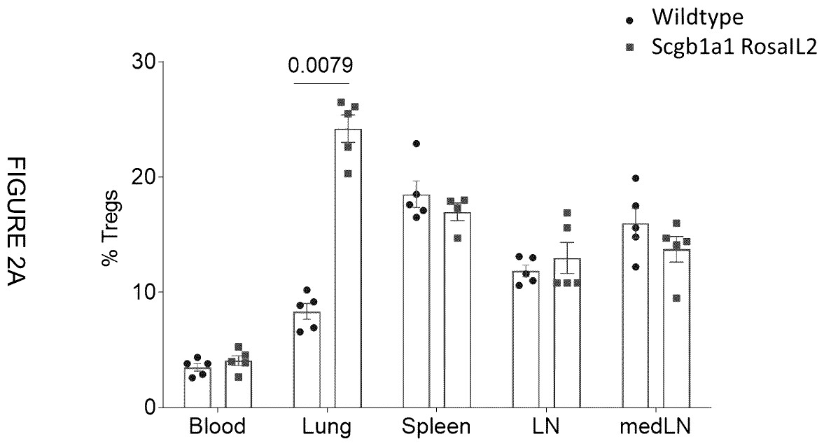

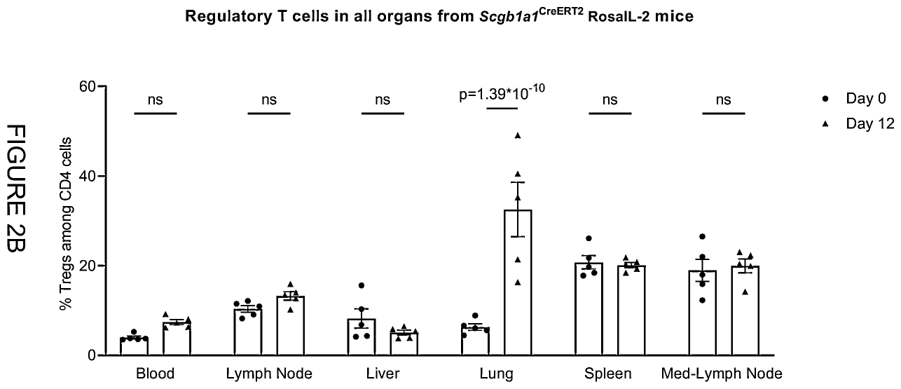

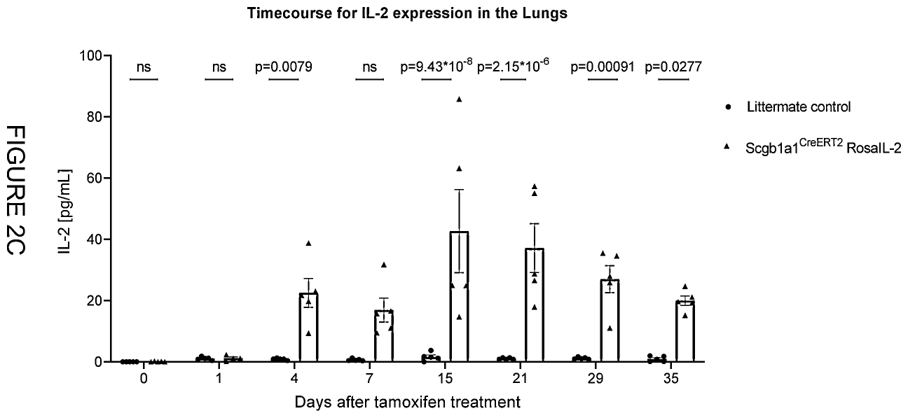

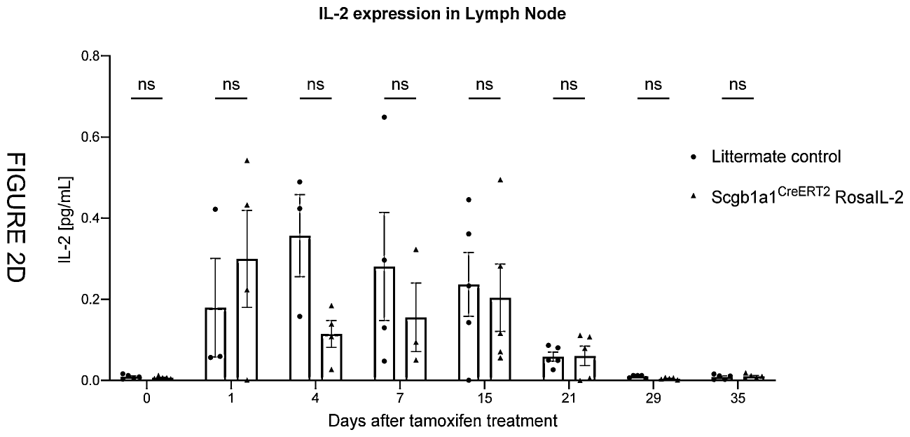

Regulatory T cells (also known as Tregs) are a subpopulation of T cells that modulate the immune system, maintain tolerance and prevent autoimmune disease. They generally suppress or downregulate the activation and/or proliferation of effector T cells and have been shown to have utility in immunosuppression. As such, regulatory T cells are highly potent cells that combine multiple immunosuppressive and regenerative capabilities and there is great interest in using exogenous regulatory T cells as a cell therapy or exogenous factors which stimulate, activate or expand endogenous regulatory T cells. The present inventors have demonstrated that regulatory T cells in the lung recirculate approximately every six weeks (FIG. 1) and therefore that modification of the homeostasis kinetics of this tissue provides a window to expand the population of these cells for immunosuppressive treatment, such as anti-inflammatory treatment, in the lung.

Thus, in one embodiment, the expanded population of regulatory T cells produced according to the methods defined herein comprises an increased anti-inflammatory potential. Such increased anti-inflammatory potential may be compared to a non-expanded population of regulatory T cells, such as a non-expanded population of regulatory T cells present in the tissue or organ, or to a population of regulatory T cells present at another location other than the tissue or organ of interest. In one embodiment, the expanded population of regulatory T cells comprises a phenotype similar to non-expanded regulatory T cells within the tissue or organ of interest or to regulatory T cells from a location other than the tissue or organ of interest. Such phenotypes may include surface marker phenotype, transcriptomic phenotype/signature (e.g. gene expression signature), gene and/or protein expression profile and cytokine expression profile. Thus, in a particular embodiment, the expanded population of regulatory T cells comprises or retains the anti-inflammatory potential of a non-expanded population of regulatory T cells or the expanded population of regulatory T cells prior to expansion. In a further embodiment, the expanded population of regulatory T cells comprises or retains the anti-inflammatory potential of a population of regulatory T cells from another location other than the tissue or organ of interest.

References herein to the phrase “in a tissue or organ” refer to a discrete location in the subject such as in a particular tissue or organ. It will be appreciated that such terms do not relate to wherein an effect is produced systemically or outside of the tissue or organ of interest, or wherein a cell type or cell population not located in the tissue or organ of interest is affected (e.g. expanded or activated). Thus, in one embodiment the population of regulatory T cells produced according to the methods defined herein is affected (e.g. expanded) in a particular tissue or organ, i.e. locally. In a further embodiment, the population of regulatory T cells is affected (e.g. expanded) in a particular tissue or organ only. In a yet further embodiment, the population of regulatory T cells located outside or not in the tissue or organ of interest is not affected (e.g. expanded). Thus, in particular embodiments, the systemic or peripheral population of regulatory T cells is not affected (e.g. expanded). In further embodiments, the population of regulatory T cells in the lymph nodes and/or spleen is not affected. In still further embodiments, the population of regulatory T cells in the blood is not affected. In yet further embodiments, the population of regulatory T cells in the liver is not affected.

Tissues or organs as defined herein comprise a discrete location of the body or of an organism. For example, the tissue or organ may comprise the lung. Thus, in a particular embodiment, the tissue or organ is the lung.

IL-2 is a key population control factor for regulatory T cells. Regulatory T cells have a naturally high turnover frequency compared to other T cells, with rapid proliferation and high apoptosis rates. IL-2 is able to increase the frequency of regulatory T cells through the induction of the anti-apoptotic protein Mcl1, which in turn reduces the Bim-dependent apoptotic rate (Pierson et al. (2013), doi:https://doi.org/10.1038.ni.2649). Increased IL-2 levels can therefore expand the size of the regulatory T cell population (Liston and Gray (2014), doi:https://doi.org/10.1038/nri3605). IL-2 delivery has been shown to be a potent anti-inflammatory agent via the expansion of this regulatory T cell population in multiple pre-clinical studies, and optimisation of IL-2 delivery is being clinically investigated. Therefore, in the context of the lung, local delivery of IL-2 will provide a high potential therapeutic for inflammation through lung-specific expansion or increase in regulatory T cell numbers, without the detrimental effects of systemic immunosuppression. In the context of treating a respiratory infection, it will be appreciated that this ability is critical since systemic immunosuppression would impede the body's ability to make antibodies, which would allow the virus to spread. The current invention therefore provides the ability to block damaging inflammation in the lung while permitting the systemic production of antibodies.

Thus, according to certain embodiments of the present invention, there is provided herein a method of expanding a population of regulatory T cells in a tissue or organ of a subject in need thereof such that wide-spread peripheral or systemic immunosuppression, which would be untenable to patients due to e.g. reduced antibody production against the infectious trigger of lung immunopathology or an increased risk of secondary infection, is avoided.

References herein to “administration” will be appreciated to refer to the providing or the making available of IL-2 at a discrete location or site of the organism, such as a particular tissue or organ. Such administration will therefore be likened with the definitions of “in a tissue or organ” as previously described herein. Thus, in one embodiment, administration of IL-2 comprises administration to or in a particular tissue or organ. In particular embodiments, administration of IL-2 comprises expression of IL-2 in a particular tissue or organ (e.g. the lung), i.e. comprises tissue- or organ-specific expression of IL-2 in said tissue or organ of said subject. In one embodiment, administration comprises expression of a gene encoding for IL-2 in a particular tissue or organ (e.g. the lung). Thus, in one embodiment, administration of IL-2 comprises tissue- or organ-specific expression of IL-2 in said tissue or organ of said subject. In a particular embodiment, the tissue- or organ-specific expression of IL-2 is in the lung. In some embodiments, the tissue- or organ-specific expression of IL-2 is maximal at 15 days after administration. In some embodiments, the tissue- or organ-specific expression of IL-2 can be detected between 4 and 35 days after administration. Thus, in one embodiment the tissue- or organ-specific expression of IL-2 can be detected at 4 days after administration. In a further embodiment, the tissue- or organ-specific expression of IL-2 can be detected for 35 days after administration. In a yet further embodiment, maximal tissue- or organ-specific expression of IL-2 can be detected at 15 days after administration. In a further embodiment, expression of IL-2 is not detectable outside the tissue or organ of interest, such as in the periphery (e.g. in the blood). In a yet further embodiment, expression of IL-2 is not detected in the lymph nodes and/or spleen. In a still further embodiment, expression of IL-2 is expression which is restricted to the particular tissue or organ of interest. In a further embodiment, expression of IL-2 is tissue- or organ-specific expression. Thus, in a particular embodiment, tissue- or organ-specific expression of IL-2 is driven by a tissue- or organ-specific promoter. In certain embodiments, administration or expression of IL-2 may be in more than one tissue or organ of interest. In one embodiment, administration or expression of IL-2 is in one, two, or more related tissues or organs (e.g. in the lung and tissues of the respiratory tract). In another embodiment, administration or expression of IL-2 is in one, two, or more tissues or organs considered not to be related.

Furthermore, references herein to “administration” and “expression” also refer to wherein IL-2 is provided to a population of cells in a tissue or organ. Such provision of IL-2 may, in one embodiment, comprise administration of IL-2 in protein or peptide form to or in the tissue or organ of interest, i.e. locally. In a further embodiment, the provision of IL-2 comprises the expression of IL-2 in the cells of the tissue or organ of interest. Thus, in a particular embodiment, expression of IL-2 comprises the cells of the tissue or organ of interest, such as those cells which make up said tissue or organ, expressing IL-2. In some embodiments, expression of IL-2 comprises epithelial cells; airway epithelial cells (such as goblet cells, ciliated cells, club cells, neuroendocrine cells (neuroendocrine bodies), basal cells, intermediate (parabasal) cells, serous cells, brush cells, special type cells with numerous intracytoplasmic membrane-bound inclusions, non-ciliated columnar cells and metaplastic cells); and alveolar cells (such as type 1 and type 2 pneumocytes, transitional type 1 and type 2 pneumocytes and cuboidal non-ciliated cells). In one embodiment, expression of IL-2 comprises airway epithelial cells, such as club cells, and/or alveolar cells, such as type 2 alveolar cells. In a particular embodiment, expression of IL-2 comprises club cells. In a further embodiment, expression of IL-2 comprises expression in cells other than the regulatory T cells which make up the expanded population of regulatory T cells produced according to the methods defined herein. Thus, in a yet further embodiment, expression of IL-2 is not in a population of regulatory T cells produced according to the methods defined herein.

In an alternative embodiment, administration or expression of IL-2 comprises introducing into the cells of the tissue or organ exogenous sequence encoding IL-2. Thus, in one embodiment, administration or expression of IL-2 comprises expression from an exogenous sequence. In a further embodiment, administration or expression of IL-2 comprises expression from a transgene. In a yet further embodiment, the transgene comprises a gene or an element encoding for IL-2. In a particular embodiment, the exogenous sequence is an IL-2 encoding sequence. In a further embodiment, the transgene comprises an IL-2 encoding sequence or gene.

In one embodiment, the exogenous sequence encoding IL-2 is in the form of a transgene comprising a tissue- or organ-specific promoter. Such tissue- or organ-specific promoters are known in the art and include promoters which drive the expression of tissue- or organ-specific genes. In one embodiment, the transgene comprises a tissue- or organ-specific promoter which specifically drives expression in the tissue or organ of interest. In a further embodiment, the transgene comprises a tissue- or organ-specific promoter which does not lead to expression in a tissue or organ other than the tissue or organ of interest. Thus, in one embodiment, the tissue- or organ-specific promoter is a lung-specific promoter. In a further embodiment, the transgene comprises a promoter which drives expression specifically in cells of the lung. In a yet further embodiment, the transgene comprises a promoter which drives expression specifically in epithelial cells, airway epithelial cells and/or alveolar cells as defined hereinbefore. In one embodiment, the transgene comprises a promoter which drives expression specifically in the lung. In a particular embodiment, the transgene comprises a promoter which drives expression specifically in airway epithelial cells, such as club cells, and/or alveolar cells, such as type 2 alveolar cells. In a further embodiment, the transgene comprises a promoter which drives expression specifically in club cells. In a yet further embodiment, the transgene comprises the surfactant protein B (SFTPB) promoter. In another embodiment, the transgene comprises the club cell-specific protein (CC10) promoter.

Thus, in one embodiment, the tissue- or organ-specific promoter is the surfactant protein B (SFTPB) promoter. In another embodiment, the tissue- or organ-specific promoter is the club cell-specific protein (CC10) promoter. In particular embodiments, the tissue- or organ-specific promoter is the SFTPB promoter and specifically drives expression in club cells and/or type 2 alveolar cells. In further embodiments, the tissue- or organ-specific promoter is the CC10 promoter and specifically drives expression in club cells.

In a further embodiment, administration or expression of IL-2 comprises a transgene which comprises an element which promotes or induces the expression of IL-2 in the presence of an exogenous compound. Such elements which promote or induce expression are known in the art and include, for example, tetracycline (Tet)-inducible systems. Tet-inducible systems provide reversible control of transcription and utilise a tetracycline-controlled transactivator (tTA) which binds tetracycline operator (TetO) sequences contained in a tetracycline response element (TRE) placed upstream of the gene/coding region of interest (and its promoter, such as a tissue-specific promoter). They may either be TetOff or TetOn systems. The TetOff system of inducible expression (also known as the tTA-dependent system) uses a tTA protein created by fusing the tetracycline repressor (TetR), found in Escherichia coli bacteria, with the activation domain of another protein, VP16, found in the Herpes Simplex Virus. The resulting tTA is able to bind TetO sequences within the TRE in the absence of tetracycline and promote expression of the downstream gene/coding region. In the presence of tetracycline, tTA binding to the TetO sequences is prevented, resulting in reduced gene expression. Conversely, the TetOn system (also known as the rtTA-dependent system) uses a reverse Tet repressor (rTetR) to create a reverse tetracycline-controlled transactivator (rtTA) protein which relies on the presence of tetracycline to promote expression. Therefore, rtTA only binds to TetO sequences within the TRE and promotes expression in the presence of tetracycline. Specific examples of TetOn systems include, but are not limited to, TetOn Advanced, TetOn 3G and the T-REx system from Life Technologies. Derivatives and analogues of tetracycline may be used with either the TetOff or TetOn systems and include, without limitation, doxycycline. Such derivatives/analogues will be appreciated to provide significant advantages compared to tetracycline such as increased stability in the case of doxycycline. Thus, in certain embodiments, the exogenous sequence encoding IL-2, such as the transgene comprising a tissue- or organ-specific promoter, further comprises a tetracycline response element (TRE). As such, in one embodiment, administration or expression of IL-2 is tetracycline-dependent or tetracycline-inducible. In a further embodiment, administration or expression of IL-2 comprises introducing into the cells of the tissue or organ exogenous sequence encoding a reverse tetracycline-controlled transactivator (rtTA). In one embodiment, the exogenous sequence encoding an rtTA comprises a tissue- or organ-specific promoter, i.e. expression of the rtTA-encoding sequence is under the control of a tissue- or organ-specific promoter as disclosed herein. Thus, in a further embodiment, the exogenous sequence encoding an rtTA comprises a promoter specific for the lung. In a yet further embodiment, expression of the rtTA-encoding sequence is under the control of a promoter specific for the lung. In a particular embodiment, the exogenous sequence encoding an rtTA comprises a promoter which drives expression specifically in airway epithelial cells, such as club cells, and/or alveolar cells, such as type 2 alveolar cells. In a further embodiment, the exogenous sequence encoding an rtTA comprises the surfactant protein B (SFTPB) promoter. In a yet further embodiment, the exogenous sequence encoding an rtTA comprises the club cell-specific protein (CC10) promoter. Such an rtTA-encoding exogenous sequence may be a separate sequence to the exogenous sequence encoding IL-2, e.g. it may be separate from the IL-2 transgene comprising a tissue- or organ-specific promoter. Alternatively, such an rtTA-encoding exogenous sequence may be comprised together with the IL-2-encoding sequence, e.g. it may be comprised in the same transgene. Thus, in some embodiments, administration or expression of IL-2 comprises a TetOn system. It will therefore be appreciated that, in one embodiment, administration or expression of IL-2 comprises the administration of tetracycline or a derivative/analogue of tetracycline, such as doxycycline.

The use of tetracycline-dependent or tetracycline-inducible administration or expression of IL-2 provides another level of control and allows the administration or expression of IL-2 to be ‘switched’ on or off. Such switching will be appreciated to be advantageous in the methods described herein by allowing the expansion of a population of regulatory T cells in a tissue or organ to be temporally controlled. For example, expression of IL-2 may be switched ‘on’ by administering tetracycline or a derivative/analogue thereof when inflammation of the lung, such as type 1 inflammation, is detected/diagnosed. Alternatively, expression of IL-2 may be switched ‘on’ following an infection, such as infection with a respiratory virus. Expression of IL-2 may then be switched ‘off’ by removal of tetracycline or a derivative/analogue thereof when inflammation, such as type 1 inflammation, is no longer detected or has reduced. Expression may also be switched ‘off’ after the subject is deemed to no longer be at risk of an infection, such as infection with a respiratory virus. Said use of tetracycline-dependent or tetracycline-inducible administration or expression of IL-2 further provides dose-dependent IL-2 administration of expression. For example, the level and/or amount of IL-2 administration or expression may be altered and/or titrated in the tissue or organ to depend on the level and/or amount of inflammation, such as type 1 inflammation, in the tissue or organ. Therefore, expression of IL-2 may be switched ‘on’ by administering a particular dose of tetracycline or a derivative/analogue thereof when inflammation of the lung, such as type 1 inflammation, is detected/diagnosed and said dose may be increased if the inflammation persists. Similarly, said dose may be decreased if the inflammation decreases following initial administration of tetracycline or a derivative/analogue thereof.

It will be appreciated that, according to embodiments wherein administration or expression of IL-2 comprises a TetOn system, the presence of a tissue- or organ-specific promoter to control expression of IL-2 may not be required. For example, expression of the rtTA-encoding sequence may be under the control of a tissue- or organ-specific promoter while expression of IL-2 is under the control of a tetracycline response element (TRE) and not a tissue- or organ-specific promoter. Thus, in one embodiment, wherein administration or expression of IL-2 is tetracycline-dependent or tetracycline-inducible, the exogenous sequence encoding an rtTA comprises a promoter specific for the lung (e.g. the SFTPB promoter or the CC10 promoter) while the exogenous sequence encoding IL-2 does not comprise a promoter specific for the lung. In a further embodiment, wherein administration or expression of IL-2 is tetracycline-dependent or tetracycline-inducible, expression of the rtTA-encoding sequence is under the control of a promoter specific for the lung (e.g. the SFTPB promoter or the CC10 promoter) while expression of the exogenous sequence encoding IL-2 is not under the control of a promoter specific for the lung.

In another embodiment, wherein administration or expression of IL-2 is tetracycline-dependent or tetracycline-inducible, both the exogenous sequence encoding an rtTA and the exogenous sequence encoding IL-2 comprise a promoter specific for the lung (e.g. the SFTPB promoter or the CC10 promoter). In a yet further embodiment, wherein administration or expression of IL-2 is tetracycline-dependent or tetracycline-inducible, expression of both the rtTA-encoding sequence and the exogenous sequence encoding IL-2 are under the control of a promoter specific for the lung (e.g. the SFTPB promoter or the CC10 promoter). In such embodiments, expression of IL-2 will be subject to a further level of control to further ensure tissue- or organ-specific administration or expression.

In one embodiment, the transgene as defined herein is introduced into the cells of the tissue or organ of interest by transduction, such as transduction using a virus or viral vector. In a particular embodiment, the transduction uses an adeno-associated virus. Thus, in one embodiment, administration of IL-2 comprises transduction, such as viral transduction. In a further embodiment, administration of IL-2 comprises adeno-associated virus transduction.

In one embodiment, transduction of the transgene as defined herein utilises a viral vector which specifically targets or infects the cells of the tissue or organ of interest. Thus, in one embodiment, transduction of the transgene as defined herein specifically targets or infects the cells of the tissue or organ of interest. According to this embodiment, it will be appreciated that transduction using a viral vector of the transgene as defined herein does not target or infect a population of regulatory T cells. In a further embodiment, transduction of the transgene as defined herein comprises a viral vector which is capable of accessing the tissue or organ of interest and is capable of crossing a barrier which may separate the tissue or organ of interest from other tissues, organs or the rest of the organism. Thus, in one embodiment, transduction comprises a viral vector capable of specifically targeting or infecting the lung. In a further embodiment, transduction comprises a viral vector capable of targeting or infecting cells of the lung.

In one embodiment, transduction comprises a lung targeting or infecting virus or viral vector. An example of a lung-targeting virus/viral vector is, but is not limited to, AAV6 as well as its variants and derivatives (e.g. AAV6.2 and AAV6.2FF). In certain embodiments, the transgene as defined herein is comprised in a viral vector, such as an adeno-associated virus vector (e.g. AAV6.2). In a further embodiment, transduction comprises the adeno-associated virus variant AAV6 and its derivatives, such as AAV6.2 and AAV6.2FF. In a yet further embodiment, transduction comprises an AAV6.2 viral vector. In a still further embodiment, transduction comprises an AAV6.2FF viral vector. In another embodiment, the transgene as defined herein is comprised in an AAV6.2 viral vector. In a further embodiment, the transgene as defined herein is comprised in an AAV6.2FF viral vector. Thus, in one embodiment, the transduction and/or the viral vector comprises AAV6.2-SFTPB-IL2, which is the AAV6.2 derivative of AAV6 comprising a transgene which contains an IL-2 encoding sequence and the lung-specific promoter, SFTPB. In another embodiment, the transduction and/or the viral vector comprises AAV6.2FF-SFTPB-IL2, which is the AAV6.2FF derivative of AAV6 comprising a transgene which contains an IL-2 encoding sequence and the lung-specific promoter, SFTPB. In a further embodiment, the transduction and/or the viral vector comprises AAV6.2-CC10-IL2, which is the AAV6.2 derivative of AAV6 comprising a transgene which contains an IL-2 encoding sequence and the lung-specific promoter, CC10. In a yet further embodiment, the transduction and/or the viral vector comprises AAV6.2FF-CC10-IL2, which is the AAV6.2FF derivative of AAV6 comprising a transgene which contains an IL-2 encoding sequence and the lung-specific promoter, CC10. Viral vectors may be used to integrate the target sequence, such as a transgene, into the host cell genome, such as the genome of a cell of the tissue or organ of interest. Thus, in certain embodiments, transduction comprises integration of the transgene as defined herein into the genome of a cell of the tissue or organ of interest such that long-term expression of the transgene in the tissue or organ is achieved. Viral vectors, such as adeno-associated viral vectors, may also be used to enable stable or long-term expression without integration of the target sequence into the host cell genome. Thus, in one embodiment, the transgene and/or target sequence are stably maintained outside the host cell genome.

References herein to a “virus” and/or “viral vector” include a virus which is non-lytic or lysogenic. Such viruses will be appreciated to achieve infection of a cell, such as a cell of the tissue or organ of interest, or introduction of a transgene into a cell without death or destruction of said cell.

It will be appreciated from the disclosures presented herein that combination of a virus or viral vector which specifically targets or infects cells of the tissue- or organ of interest (e.g. a lung-specific virus or viral vector) and a promoter which drives expression specifically in cells of the tissue or organ of interest, provides exceptional specificity. Such specificity provides a so-called ‘dual lock’, restricting both the cells into which the transgene is targeted or infected and in which cells the transgene is expressed. Thus, in one embodiment, the combination of a tissue- or organ-specific viral vector and tissue- or organ-specific promoter as defined herein provides that only those cells of the tissue or organ of interest comprise the transgene as defined herein and only those cells of the tissue or organ of interest are capable of expressing said transgene. In a further embodiment, the combination of a tissue- or organ-specific viral vector and tissue- or organ-specific promoter as defined herein provides that only those cells of the tissue or organ of interest comprise an IL-2-encoding gene and only those cells of the tissue or organ of interest are capable of expressing said gene.

In a yet further embodiment, the combination of a tissue- or organ-specific viral vector and tissue- or organ-specific promoter as defined herein together with an inducible element, such as a tetracycline-inducible element, provides that only those cells of the tissue or organ of interest comprise the transgene as defined herein and only those cells of the tissue or organ of interest are capable of expressing said transgene when an activator of the inducible element is administered (e.g. tetracycline or doxycycline). In one embodiment, the combination of a tissue- or organ-specific viral vector and tissue- or organ-specific promoter as defined herein together with an inducible element, such as a tetracycline-inducible element, provides that only those cells of the tissue or organ of interest comprise an IL-2-encoding gene and only those cells of the tissue or organ of interest are capable of expressing said gene when an activator of the inducible element is administered (e.g. tetracycline or doxycycline). In a further embodiment, said combination provides that only those cells of the tissue or organ of interest comprise an inducible IL-2-encoding gene and only those cells of the tissue or organ of interest are capable of expressing a reverse tetracycline-controlled transactivator (rtTA) which leads to the expression of IL-2 when an activator of the inducible element is administered (e.g. tetracycline or doxycycline).

Administration of IL-2 as defined herein may further comprise administration of IL-2 directly to the tissue or organ of interest. Examples of direct administration include injection directly into the tissue or organ of interest or utilise a suitable delivery device. Such delivery devices are known in the art and, according to the present disclosures, allow for the controlled and/or sustained administration of IL-2 for the duration of treatment (e.g. chronically or for duration of treatment of an acute inflammatory disease or disorder). In the case of administration/delivery to the lung, inhalation and/or intranasal delivery may be utilised, for example using an inhaler or nebuliser or by spraying an atomised solution comprising the IL-2 and/or viral vector described herein.

The duration of IL-2 administration as defined herein can be altered to depend on the treatment and the characteristics of the particular inflammatory condition or disease to be treated by the methods described herein. For example, administration of IL-2 may be chronic. Alternatively, administration of IL-2 may be for the duration of treatment for the disease or disorder, such as in the treatment of an acute inflammatory condition or infection. Thus, in certain embodiments, the duration of administration or expression of IL-2 depends on the disease or disorder to be treated or on the duration of the treatment. In one embodiment, administration or expression of IL-2 is acute. In another embodiment, administration or expression of IL-2 is chronic.

It will be appreciated that IL-2 and a targeting moiety specific for a tissue or organ may be combined or co-administered. Therefore, the administration of IL-2 may comprise expression of IL-2 in the tissue or organ of interest as defined herein (e.g. tissue- or organ-specific expression) and can be combined with a targeting moiety specific for the tissue or organ of the subject. Furthermore, administration of IL-2 may comprise administration of IL-2 in protein or peptide form and can be combined with a targeting moiety specific for the tissue or organ of the subject.

References herein to the term “targeting moiety” refer to any moiety that provides for the tissue- or organ-specific administration or expression of IL-2 as defined herein. Furthermore, said targeting moiety will be appreciated to provide for the localised administration or expression of IL-2 as defined herein.

Thus, in one embodiment of the present invention, the methods defined herein comprise administration of a targeting moiety specific for the tissue or organ of the subject. In a further embodiment, the targeting moiety specific for the tissue or organ of the subject localises IL-2 in or to the tissue or organ of interest. Thus, in one embodiment, the targeting moiety specific for the tissue or organ of the subject localises IL-2 only in or to the tissue or organ of interest. In a further embodiment, the targeting moiety specific for the tissue or organ of the subject prevents localisation of IL-2 to other tissues or organs other than the tissue or organ of interest, or localises IL-2 away from tissues or organs other than the tissue or organ of interest. In another embodiment, the targeting moiety provides for expression of IL-2 in the tissue or organ of interest. Thus, in one embodiment, the targeting moiety specific for the tissue or organ of the subject provides for expression of IL-2 only in the tissue or organ of interest. Such references herein to “in the tissue or organ of interest” further include wherein said effect is in the cells which make up said tissue or organ (e.g. epithelial cells, airway epithelial cells and/or alveolar cells).

In one embodiment, the targeting moiety specific for the tissue or organ of the subject is a virus or viral vector as defined herein. In a further embodiment, said virus or viral vector specifically targets or infects the tissue or organ of interest or specifically targets or infects cells of the tissue or organ of interest. Thus, according to this embodiment, said targeting moiety specific for the tissue or organ of interest which is a virus or viral vector that does not target or infect cells in other tissues or organs other than the tissue or organ of interest, or target or infect cells which make up a tissue or organ other than the tissue or organ of interest. Also according to this embodiment, it will be appreciated that said targeting moiety specific for the tissue or organ as defined herein does not target or infect a population of regulatory T cells. In a further embodiment, the targeting moiety specific for the tissue or organ of a subject as defined herein comprises a virus or viral vector which is capable of accessing the tissue or organ of interest and is capable of crossing a barrier which separates the tissue or organ of interest from other tissues, organs or the rest of the subject. Thus, in one embodiment, the targeting moiety specific for a tissue or organ comprises a virus or viral vector capable of specifically targeting or infecting the lung. In a further embodiment, the targeting moiety specific for a tissue or organ comprises a virus or viral vector capable of targeting or infecting epithelial cells, airway epithelial cells and/or alveolar cells. In a particular embodiment, the targeting moiety specific for a tissue or organ comprises a virus or viral vector capable of targeting or infecting club cells. In another embodiment, the targeting moiety specific for a tissue or organ comprises a virus or viral vector capable of targeting or infecting type 2 alveolar cells.

In one embodiment, the targeting moiety specific for a tissue or organ comprises an adeno-associated virus. In a further embodiment, the targeting moiety is an adeno-associated virus selected from: AAV6 and its variants/derivatives, such as AAV6.2 and AAV6.2FF. In a particular embodiment, the targeting moiety specific for a tissue or organ comprises the adeno-associated virus variant AAV6.2. In another embodiment, the targeting moiety specific for a tissue or organ comprises the adeno-associated virus variant AAV6.2FF. In certain embodiments, the transgene as defined herein is comprised in a targeting moiety specific for a tissue or organ, such as an adeno-associated virus vector, which is comprised within an adeno-associated virus as defined herein. In one embodiment, the transgene as defined herein is comprised in an adeno-associated virus selected from: AAV6 and its variants/derivatives, such as AAV6.2 and AAV6.2FF. Thus, in one embodiment, the transgene which contains an IL-2 encoding sequence and the lung-specific promoter, SFTPB, is comprised in the AAV6 derivative AAV6.2 virus/viral vector and the virus/viral vector is AAV6.2-SFTPB-IL2. In another embodiment, the transgene which contains an IL-2 encoding sequence and the lung-specific promoter, CC10, is comprised in the AAV6.2 virus/viral vector and the virus/viral vector is AAV6.2-CC10-IL2. In a further embodiment, the transgene which contains an IL-2 encoding sequence and the lung-specific promoter, SFTPB, is comprised in the AAV6 derivative AAV6.2FF virus/viral vector and the virus/viral vector is AAV6.2FF-SFTPB-IL2. In a yet further embodiment, the transgene which contains an IL-2 encoding sequence and the lung-specific promoter, CC10, is comprised in the AAV6.2FF virus/viral vector and the virus/viral vector is AAV6.2FF-CC10-IL2.

According to a further aspect of the invention, there is provided a method for the expansion of a population of regulatory T cells in a tissue or organ in vivo. Embodiments of the present aspect will be appreciated to be equivalent and comparable to all embodiments previously described herein. Thus, in certain embodiments, the term “of a subject” as described herein is synonymous with “in vivo”.

In one embodiment, the method for expanding a population of regulatory T cells in a tissue or organ in vivo comprises administration of IL-2 as described herein. In a further embodiment, the method for expanding a population of regulatory T cells in a tissue or organ in vivo comprises administration of a targeting moiety specific for the tissue or organ of a subject in vivo. In one embodiment, the administration of IL-2, which may comprise expression of IL-2, is combined with a targeting moiety specific for a tissue or organ in vivo. In a further embodiment, the method for expanding a population of regulatory T cells in a tissue or organ in vivo comprises a virus or viral vector which comprises an IL-2-encoding gene. In one embodiment, said virus or viral vector is capable of targeting or infecting a tissue or organ of interest. In a particular embodiment, said virus or viral vector capable of targeting or infecting a tissue or organ of interest, specifically targets or infects cells of a tissue or organ of interest. In a further embodiment, the method for expanding a population of regulatory T cells in a tissue or organ in vivo comprises a virus or viral vector which comprises a tissue- or organ-specific promoter. Thus, in a particular embodiment, the method for expanding a population of regulatory T cells in a tissue or organ in vivo comprises administration of a targeting moiety specific for the tissue or organ of interest, wherein said targeting moiety is a virus or viral vector which specifically targets or infects the lung. In a further embodiment, the method for expanding a population of regulatory T cells in a tissue or organ in vivo comprises administration of a targeting moiety specific for the tissue or organ of interest, wherein said targeting moiety is specific for epithelial cells, airway epithelial cells, and/or alveolar cells. In a yet further embodiment, the targeting moiety specific for a tissue or organ of interest is specific for club cells and/or type 2 alveolar cells. In another embodiment, the method for expanding a population of regulatory T cells in a tissue or organ in vivo comprises administration of a lung-specific virus or viral vector containing the transgene as defined herein, such as administration of AAV6.2-SFTPB-IL2, AAV6.2FF-SFTPB-IL2, AAV6.2-CC10-IL2 or AAV6.2FF-CC10-IL2 as defined herein.

According to one aspect of the invention, there is provided a population of regulatory T cells expanded according to or obtained by the methods described herein. Thus, in one embodiment, there is provided an expanded population of regulatory T cells which have been expanded in a tissue or organ of a subject by administration of IL-2 and a targeting moiety specific for said tissue or organ.

Pharmaceutical Compositions

According to one aspect of the invention, there is provided a pharmaceutical composition comprising IL-2 and a targeting moiety specific for a tissue or organ of a subject, wherein said targeting moiety is specific for the lung.

In one embodiment, the pharmaceutical composition comprises IL-2 which promotes the expansion of a population of regulatory T cells. In a yet further embodiment, the pharmaceutical composition comprises a targeting moiety specific for a tissue or organ of a subject. In one embodiment, the targeting moiety specific for a tissue or organ of a subject is a virus or viral vector which specifically targets or infects cells of the tissue or organ and drives tissue- or organ-specific expression of IL-2 as described herein. Thus, according to this aspect of the invention, there is provided a pharmaceutical composition comprising a tissue- or organ-specific viral vector which expands a population of regulatory T cells in said tissue or organ of the subject. In particular embodiments, the pharmaceutical composition expands a population of regulatory T cells specifically or locally in a tissue or organ of interest in a subject.

In one embodiment, the pharmaceutical composition as defined herein comprises a targeting moiety capable of crossing a barrier which separates a tissue or organ of interest from other tissues or organs or from the rest of the organism. Thus, in one embodiment, the pharmaceutical composition as defined herein comprises an adeno-associated virus selected from AAV6 and its variants/derivatives, such as AAV6.2 and AAV6.2FF. In a further embodiment, the pharmaceutical composition as defined herein comprises the adeno-associated virus variant AAV6.2. In a yet further embodiment, the pharmaceutical composition as defined herein comprises the adeno-associated virus variant AAV6.2FF. In a further embodiment, the viral vector comprised in the pharmaceutical composition as defined herein comprises a gene, such as a transgene, which encodes for IL-2. In a yet further embodiment, the transgene comprised in the viral vector of the pharmaceutical composition further comprises a tissue- or organ-specific promoter as defined herein.

Thus, in certain embodiments, the pharmaceutical composition as defined herein comprises a tissue- or organ-specific virus or viral vector capable of targeting or infecting cells of the tissue or organ of interest, comprising an IL-2-encoding gene, expression of which is driven by a tissue- or organ-specific promoter. In one particular embodiment, the pharmaceutical composition as defined herein comprises a viral vector, such as an adeno-associated virus (e.g. AAV6 and its variants/derivatives, such as AAV6.2 and AAV6.2FF), which specifically targets or infects the lung, such as cells of the lung (e.g. epithelial cells, airway epithelial cells and/or alveolar cells), which comprises an IL-2-encoding gene, expression of which is driven by a tissue- or organ-specific promoter. In a further embodiment, the pharmaceutical composition as defined herein comprises the adeno-associated virus AAV6.2, which comprises an IL-2-encoding gene, expression of which is driven locally in the lung or in cells of the lung by a SFTPB promoter. In another embodiment, the pharmaceutical composition comprises the adeno-associated virus AAV6.2FF, which comprises an IL-2-encoding gene, expression of which is driven locally in the lung or in cells of the lung by a SFTPB promoter.

In a further embodiment, the pharmaceutical composition comprises the adeno-associated virus AAV6.2, which comprises an IL-2-encoding gene, expression of which is driven locally in the lung or in cells of the lung by a CC10 promoter. In yet a further embodiment, the pharmaceutical composition comprises the adeno-associated virus AAV6.2FF, which comprises an IL-2-encoding gene, expression of which is driven locally in the lung or in cells of the lung by a CC10 promoter. Thus, in one embodiment, the pharmaceutical composition comprises AAV6.2-SFTPB-IL2. In another embodiment, the pharmaceutical composition comprises AAV6.2FF-SFTPB-IL2. In a further embodiment, the pharmaceutical composition comprises AAV6.2-CC10-IL2. In a yet further embodiment, the pharmaceutical composition comprises AAV6.2FF-CC10-IL2.

According to some embodiments, the pharmaceutical composition, in addition to a tissue- or organ-specific virus or viral vector as defined herein, further comprises one or more pharmaceutically acceptable excipients.

Generally, the present pharmaceutical compositions will be utilised with pharmacologically appropriate excipients or carriers. Typically, these excipients or carriers include aqueous or alcoholic/aqueous solutions, emulsions or suspensions, including saline and/or buffered media. Parenteral vehicles include sodium chloride solution, Ringer's dextrose, dextrose and sodium chloride and lactated Ringer's. Suitable physiologically-acceptable adjuvants, if necessary to keep a composition comprising the targeting moiety specific for a tissue or organ as defined herein in a discrete location (e.g. within a tissue or organ of interest), may be chosen from thickeners such as carboxymethylcellulose, polyvinylpyrrolidone, gelatine and alginates. Intravenous vehicles include fluid and nutrient replenishers and electrolyte replenishers, such as those based on Ringer's dextrose. Excipients, carriers and vehicles suitable for the delivery of a pharmaceutical composition by intranasal administration, inhalation or using a nebuliser are known and will be appreciated to find particular utility with the pharmaceutical compositions defined herein. Preservatives and other additives, such as antimicrobials, antioxidants, chelating agents and inert gases, may also be present (Mack (1982) Remington's Pharmaceutical Sciences, 16^{th} Edition).

Therapeutic Uses and Methods

It will be appreciated from the disclosures presented herein that the method of expanding a population of regulatory T cells, pharmaceutical compositions and methods of treatment of the present invention will find particular utility in the treatment and/or amelioration of diseases or disorders mediated by inflammation and/or in the reduction of inflammation. It will be further appreciated that a population of regulatory T cells expanded according to the methods and disclosures presented herein will also find utility in the treatment and/or amelioration of diseases or disorders mediated by inflammation and/or in the reduction of inflammation.

Thus, according to one aspect of the invention, there is provided a method for expanding a population of regulatory T cells in a tissue or organ of a subject for use in the treatment and/or amelioration of a disease or disorder mediated by inflammation, wherein said tissue or organ is the lung. In another aspect of the invention, there is provided a method for expanding a population of regulatory T cells in a tissue or organ of a subject for use in the reduction of inflammation, wherein said tissue or organ is the lung. In a further aspect of the invention, there is provided a method for expanding a population of regulatory T cells in a tissue or organ of a subject for use in the treatment and/or amelioration of an autoimmune disease, wherein said tissue or organ is the lung.

In another aspect of the invention, there is provided a population of expanded regulatory T cells in a tissue or organ of a subject produced according to the methods defined herein for use in the treatment and/or amelioration of a disease or disorder mediated by inflammation or for use in the reduction of inflammation. Such diseases or disorders may include inflammatory conditions, autoimmune diseases and/or diseases associated with transplant, such as transplant rejection or graft vs. host disease. In one embodiment, the expanded population of regulatory T cells in a tissue or organ of a subject produced according to the methods defined herein has been expanded by administration of IL-2 and a targeting moiety specific for said tissue or organ. In a further embodiment, the population of expanded regulatory T cells in a tissue or organ of a subject produced according to the methods defined herein has been expanded by tissue- or organ-specific expression of IL-2 as defined herein. In another embodiment, the population of expanded regulatory T cells in a tissue or organ of a subject has been expanded by tissue- or organ-specific expression of IL-2 promoted or induced by an inducible element, such as a tetracycline-inducible element. In a yet further embodiment, the population of expanded regulatory T cells in a tissue or organ of a subject produced according to the methods defined herein is for use in the treatment and/or amelioration of a disease or disorder of the lung. In one embodiment, the population of expanded regulatory T cells in a tissue or organ of a subject produced according to the methods defined herein is for use in the treatment and/or amelioration of the lung. In a yet further embodiment, the population of expanded regulatory T cells in a tissue or organ of a subject produced according to the methods defined herein is for use in the treatment and/or amelioration of type 1 inflammation. In certain embodiments, the population of expanded regulatory T cells in a tissue or organ of a subject produced according to the methods defined herein is for use in the treatment and/or amelioration of inflammation in the lung. Thus, according to one embodiment, the inflammation as defined herein is inflammation of the lung. In a further embodiment, inflammation of the lung is due to a respiratory disease or disorder. Thus, in one embodiment, the inflammation in the lung is due to a respiratory disease or disorder. In a further embodiment, the population of expanded regulatory T cells in a tissue or organ of a subject produced according to the methods defined herein is for use in the treatment and/or amelioration of inflammation of the lung, wherein the inflammation is caused by a respiratory infection or the respiratory disease or disorder is a respiratory infection, such as infection with influenza, a corona virus or a novel emerging virus. In an alternative embodiment, the inflammation is caused by a non-infectious disease or disorder or the respiratory disease or disorder is non-infectious, such as chronic obstructive pulmonary disease (COPD). Another example is an autoimmune disease or disorder and/or wherein the inflammation is due to an autoimmune disease or disorder.

According to a further aspect of the invention, there is provided a method of treating a disease or disorder mediated by inflammation and/or for the reduction of inflammation, wherein said method either comprises a method as defined herein or administering to a subject in need thereof a pharmaceutical composition comprising IL-2 and a targeting moiety specific for a tissue or organ of a subject as defined herein. In one embodiment, said method of treatment comprises administering a virus or viral vector comprising a gene encoding IL-2 as defined herein to a subject in need thereof. In one embodiment, the method of treatment as defined herein, comprises administering to a subject in need thereof a virus or viral vector which specifically targets or infects a tissue or organ affected by a disease or disorder mediated by inflammation or affected by inflammation. In certain embodiments, the method of treatment as defined herein, further comprises administering to a subject in need thereof a virus or viral vector comprising a gene encoding IL-2, expression of which is driven by a tissue- or organ-specific promoter. In a further embodiment, the method of treatment as defined herein comprises administering to a subject in need thereof a virus or viral vector comprising a gene encoding IL-2, expression of which is driven by a tissue- or organ-specific promoter and an inducible element, such as a tetracycline-inducible element. In an alternative embodiment, the method of treatment comprises administering to a subject a virus or viral vector comprising a gene encoding IL-2, expression of which is driven by an inducible element, such as a tetracycline-inducible element, under the control of a tissue- or organ-specific promoter. In further embodiments, the method of treatment as defined herein comprises administering to a subject in need thereof a virus or viral vector comprising a gene encoding IL-2, expression of which is driven by a tissue- or organ-specific promoter, such as administering AAV6.2-SFTPB-IL2, AAV6.2FF-SFTPB-IL2, AAV6.2-CC10-IL2 or AAV6.2FF-CC10-IL2.

In certain embodiments, said subject in need thereof is suffering from a disease or disorder mediated by inflammation. In further embodiments, the subject in need thereof is suffering from inflammation. In yet further embodiments, the subject in need thereof is suffering from type 1 inflammation. In other embodiments, the subject in need thereof is suffering from an autoimmune disease or disorder. In one embodiment, said disease or disorder is a disease or disorder of the lung. In a further embodiment, said disease or disorder is a respiratory disease or disorder. In a further embodiment, said inflammation or respiratory disease or disorder comprises type 1 inflammation. In another embodiment, said inflammation is caused by a respiratory infection or the respiratory disease or disorder is a respiratory infection, such as an influenza or corona virus infection, or a novel emerging respiratory virus. In an alternative embodiment, said inflammation is caused by a non-infectious disease or disorder or the respiratory disease or disorder is non-infectious, such as chronic obstructive pulmonary disease (COPD).

### EXAMPLES

### Example 1: Parabiosis Studies to Calculate Steady-State Population Exchange

Parabiosis studies allow the calculation of steady-state population exchange. Through parabiosis of Foxp3^{Thy1.1}CD45.1 mice with Foxp3^{Thy1.1}CD45.2 mice and high-dimensional flow cytometry analysis at five successive time-points (FIG. 1A), the kinetics of blood and lung tissue normalisation for Tregs could be monitored. Unlike the blood, where normalisation occurred within days, exchange-rate calculations determined that CD69⁻ CD4 T cells had a median lung dwell-time of ˜2-3 weeks, while the CD69⁺ counterpart had a median lung dwell-time of ˜7 weeks (FIG. 1B).

### Example 2: Transgenic Mouse Model for Proof-of-Principle Luna-Specific Regulatory T Cell Expansion

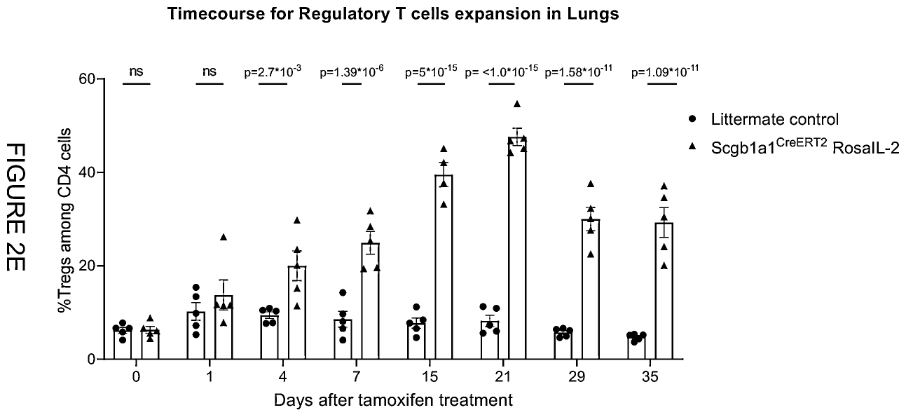

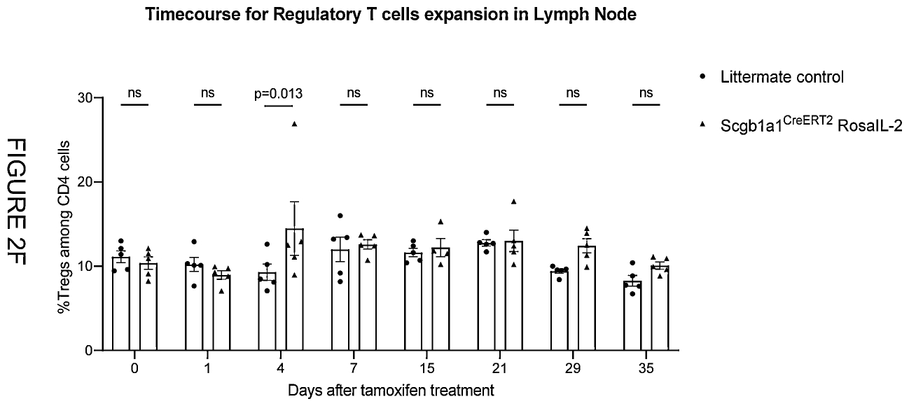

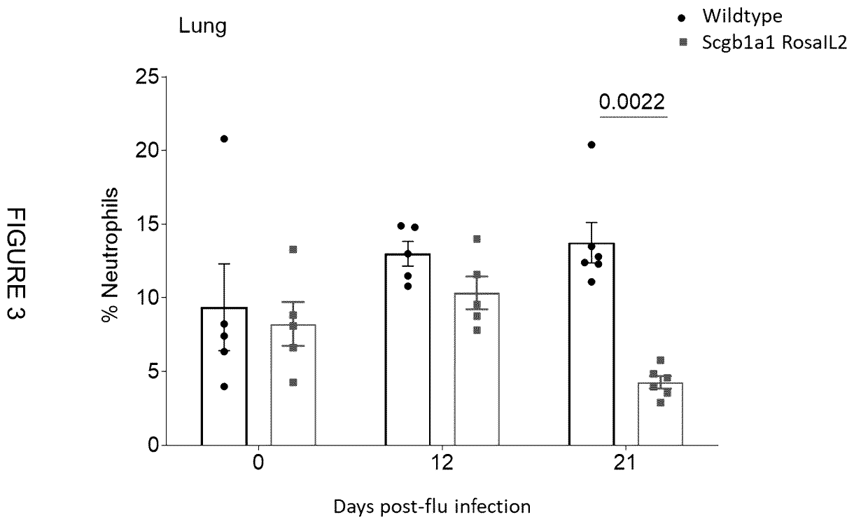

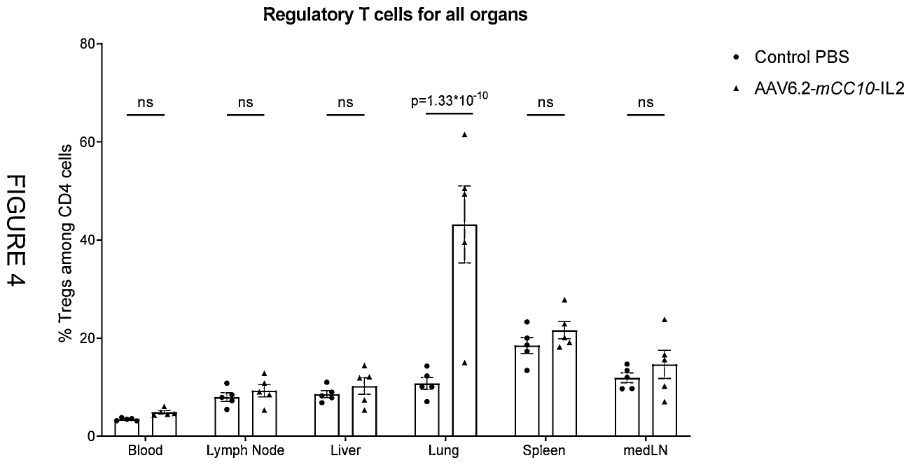

Based on the lung-dwell time of ˜7 weeks, it was reasoned that the lung-resident regulatory T cell population could be expanded by local provision of IL-2. To test this possibility a genetic switch for IL-2 expression was developed, where IL-2 could be switched on in lung tissue only. Using the constitutive Rosa26 promoter and a floxed-stop expression system, a system where Cre expression would induce cell lineage-specific IL-2 expression was created. The weak endogenous Rosa26 promoter was used to ensure that IL-2 expression remained at physiological levels; indeed, transgenic IL-2 expression in this system is only ˜5% of the expression level of native IL-2 production by conventional CD4 T cells (data not shown). To induce lung-specific expression this transgene was crossed to the Scgb1a1-ERT2^{Cre} transgenic mouse line, enabling tamoxifen-induced recombination in bronchiolar non-ciliated club cells. Scgb1a1-ERT2^{Cre} RosaIL2 mice were treated with tamoxifen and assessed for regulatory T cell numbers and a lung-specific expansion of the population was observed (FIGS. 2A and 2B). No major alterations were observed in regulatory T cell populations in the blood, secondary lymphoid organs or other non-lung tissues (FIGS. 2A and 2B).

In order to determine the dynamics of IL-2 expression in lung, lymph node tissues were taken from Scgb1a1-ERT2^{Cre} RosaIL2 mice treated with tamoxifen after 0, 1, 4, 7, 15, 21, 29 and 35 days of administration and analysed by quantitative PCR (qPCR). Data is shown in FIGS. 2C and 2D and demonstrates lung-specific expression of IL-2 in Scgb1a1-ERT2^{Cre} RosaIL2 mice compared to littermate controls (FIG. 2C). Expression is detected from day 4 after administration and remains detectable at the last day of analysis, day 35. The peak of expression is at day 15 after tamoxifen administration. No significant difference in IL-2 expression in the lymph nodes of Scgb1a1-ERT2^{Cre} RosaIL2 mice compared to littermate controls (FIG. 2D).

FIG. 2E demonstrates that the maximum increase in regulatory T cells is seen in the lung at day 21 after lung-specific IL-2 expression. An expanded proportion of Tregs in the lung is detected from day 4 after tamoxifen administration and remains elevated compared to littermate controls at the last day of analysis, day 35. This correlates with the dynamics of IL-2 expression seen in FIG. 2C with a slight to-be expected delay in cell numbers. No significant changes were seen in the percentage of Tregs in the lymph nodes of mice administered with tamoxifen throughout the time course (FIG. 2F).

This data demonstrates that local provision of IL-2 is capable of specifically expanding the lung regulatory T cell population, without expanding peripheral numbers, and that IL-2 can be expressed locally in the lung with no expression in peripheral tissues, such as in the lymph nodes.

### Example 3: Expanded Lung Regulatory T Cells Reduces Inflammatory Infiltrate During Flu Infection

In order to assess the protective effects of lung-specific regulatory T cell expansion on respiratory infection, Scgb1a1-ERT2^{Cre} RosaIL2 mice were infected with mouse flu via the intranasal route. Unlike wildtype mice, which exhibited a chronic increase in inflammatory neutrophil infiltrate in the lung after infection, Scgb1a1-ERT2^{Cre} RosaIL2 mice demonstrated a reduction in both scale and duration of disease (FIG. 3).

These results suggest that lung-targeted IL-2 administration/delivery has the potential to reduce the immunopathology of respiratory infections. Together with the above data showing lung-specific expansion of regulatory T cells, this demonstrates that such potential to reduce immunopathology is achieved without increasing the systemic regulatory T cell burden.

### Example 4: Intranasal Administration of AAV6.2-mCC10-IL2 Results in Local Expansion of Regulatory T Cells in the Lungs

To test the ability for specific and local delivery/expression of IL-2 using a viral vector, mice were administered intranasally with AAV6.2-mCC10-IL2 as described hereinbefore or control PBS and the immune cells in the lung analysed 14 days after administration. Compared to mice administered with control PBS, those given intranasal AAV6.2-mCC10-IL2 showed a significantly increased proportion of Tregs in the lung (FIG. 4). This increase was not seen in other tissues, such as lymph nodes, or in the periphery/blood.

This data therefore demonstrates that IL-2 may be delivered locally to the lung using a viral vector such as AAV6.2, resulting in an expanded Treg population specifically in the lung. Such lung-specific expansion is consistent with the use of both a lung-targeting moiety and the lung-specific promoter CC10.

1. A method of expanding a population of regulatory T cells in a tissue or organ of a subject in need thereof, wherein said method comprises administration of IL-2 and a targeting moiety specific for said tissue or organ, and wherein said tissue or organ is the lung.

2. The method of claim 1, wherein administration of IL-2 comprises tissue- or organ-specific expression of IL-2 in said tissue or organ of said subject.

3. The method of claim 2, wherein the tissue- or organ-specific expression of IL-2 is in the lung, and/or wherein tissue- or organ-specific expression of IL-2 is driven by a tissue- or organ-specific promoter.

4. (canceled)

5. The method of claim 3, wherein the tissue- or organ-specific promoter is a lung-specific promoter, such as the surfactant protein B (SFTPB) promoter or club cell-specific protein (CC10) promoter.

6. The method of claim 2, wherein the tissue- or organ-specific expression of IL-2 comprises an inducible element which promotes or induces the expression of IL-2 in the presence of an exogenous compound.

7. The method of claim 6, wherein the inducible element is a tetracycline-dependent or tetracycline-inducible element, such as a tetracycline response element (TRE).

8. The method of claim 7, wherein the tissue- or organ-specific expression of IL-2 comprises the administration of tetracycline or a derivative/analogue of tetracycline, such as doxycycline.

9. The method of claim 1, wherein administration of IL-2 comprises an exogenous IL-2 encoding sequence.

10. The method of claim 9, wherein the exogenous IL-2 encoding sequence further comprises a sequence encoding a reverse tetracycline-controlled transactivator (rtTA).

11. The method of claim 1, wherein said targeting moiety specific for the lung comprises a viral vector.

12. The method of claim 11, wherein the viral vector is an adeno-associated virus selected from AAV6 and its variants and derivates, in particular AAV6.2 and AAV6.2FF.

13. (canceled)

14. A pharmaceutical composition comprising IL-2 and a targeting moiety specific for a tissue or organ of a subject, wherein said targeting moiety is specific for the lung.

15. The pharmaceutical composition of claim 14, wherein the targeting moiety specific for the lung comprises a viral vector.

16. The pharmaceutical composition of claim 15, wherein the viral vector is an adeno-associated virus selected from AAV6 and its variants and derivates, in particular AAV6.2 or AAV6.2FF.

17. (canceled)

18. A method of treating a disease or disorder mediated by inflammation and/or for the reduction of inflammation, wherein said method comprises administering the pharmaceutical composition according to claim 14 to a subject in need thereof.

19. The method according to claim 18, wherein the inflammation is inflammation of the lung, or wherein the disease or disorder is a respiratory disease or disorder.

20. (canceled)

21. The method according to claim 19, wherein the inflammation or respiratory disease or disorder comprises type 1 inflammation, and/or wherein the inflammation is caused by a respiratory infection or the respiratory disease or disorder is a respiratory infection, such as an influenza, a corona virus infection, or a novel emerging virus.

22. (canceled)

23. The method according to claim 19, wherein the inflammation is caused by a non-infectious disease or disorder or the respiratory disease or disorder is non-infectious, such as chronic obstructive pulmonary disease (COPD).

24. The method of claim 2, wherein tissue- or organ-specific expression of IL-2 in the lung comprises an exogenous IL-2 encoding sequence.

25. The method of claim 24, wherein the exogenous IL-2 encoding sequence further comprises a sequence encoding a reverse tetracycline-controlled transactivator (rtTA).
